(19) United States
(12) Patent Application Publication
(10) Pub. No.: US 2006/0018954 A1
(43) Pub. Date: Jan. 26, 2006

(54) Magnesium-bearing wound-covering material

(75) Inventor: Marc Kuttler, Berlin (DE)

Correspondence Address: HAHN LOESER & PARKS, LLP, One GOJO Plaza, Suite 300, AKRON, OH, 44311-1076, US

(73) Assignee: BIOTRONIK VI Patent AG, Baar (CH)

(21) Appl. No.: 11/185,142

(22) Filed: Jul. 20, 2005

(30) Foreign Application Priority Data
Jul. 20, 2004 (DE) 102004 035 905.9

(51) Int. Cl. A61L 15/00; A61K 33/06

(52) U.S. Cl. 424/445; 424/682

(57) ABSTRACT

The invention concerns a biocompatible and bioabsorbable wound covering material. The object of the present invention is to improve wound care by a biocompatible, biodegradable wound covering material. In that respect body-specific defense and healing mechanisms are to be assisted and antimicrobial and antiproliferative effects are to be achieved. That is attained in that the wound covering material contains elementary magnesium.

### BACKGROUND OF THE INVENTION

The invention concerns wound covering materials which can be used for the treatment of infected wounds, other inflammatory skin diseases or for the preventive protection of wound infections and which promote the healing process.

The treatment and healing of infected or infection-endangered wounds of a human being or an animal—caused by injuries, burns, operative interventions (acute wounds), allergic reactions or other skin diseases and in particular also chronic wounds—is a primary aim of modern medical technology. Excessive colonization of the wound environment with microorganisms such as for example bacteria of the genera Pseudomonas or Staphylococcus can lead to a massive disturbance in the healing process to the extent of being lethal. It is not possible to restrict the danger to a few species as many of the microorganisms are to be viewed as opportunistic pathogens.

A large number of methods are known for removing pathogens from infected tissue or killing them off.

For example, the use of antibiotics which are applied to the wound administered orally or intravenously is wide-spread. The wide-spread use of antibiotics, however, has resulted in the creation of resistances on the part of many pathogens. Thus, in the meantime about 23% of all Staphylococcus strains have become resistant to one or more conventional one or more antibiotics such as, for example, penicillin.

Another possible way of wound care is mechanical cleaning with Ringer's solution, a very time-consuming method which also delays the healing process.

The use of oxidants (for example iodine tincture) or antiseptics (for example silver sulfadiazine) is also known for the antimicrobial therapy of wounds or for prevention generally. Metal ions, in particular precious metal ions such as gold and silver ions have been used for many years in wound care for avoiding and combating infections. The universally known silver nitrate is to be particularly emphasized. Thus for example 5% silver nitrate solution has been used from time to time for the treatment of second degree burns, which has to be applied to the area of the wound up to 12 times per day in order to afford sufficient active ions. When using silver-bearing creams (for example silver sulfadiazine) once again the residues have to be regularly removed in order to avoid allergic reactions.

Many different forms are known for the use of silver compounds in wound treatment such as powders, flakes, coatings, foils, fibers, sheets, paper or plastic materials.

Nowadays hydrophobized carrier materials are also used in modern wound treatment in order to prevent the wound from drying out and thus to promote the natural procedures involved in wound healing. In this case also, silver compounds are used because of the antimicrobial effect.

In regard to all those forms however it is still to be noted that they involve direct contact between the wound tissue and silver ions and thus healthy cell growth is also adversely influenced while the wound is healing.

The wound covering materials which are used in relation to wound care and which inter alia protect the wound from external influences and further soiling/contamination are usually non-absorbable materials which may not remain in the wound. Such wound coverings or dressings which are in contact with the wound frequently stick to the wound. Removal/replacement of the wound covering is traumatic for the patient, it delays the healing process by virtue of the wound being torn open again, and it increases the risk of renewed infection.

In the meantime bioresorbable materials have begun to be developed, which can remain in the wound and which avoid the material growing into/sticking to the other wound covering layers. They also serve as carriers as antimicrobial substances, they are intended to fill up the wound and they protect it until it is sufficiently healed. Such a bioresorbable material is for example collagen which is used inter alia in the form of beads (DE 697 14 226 T2). Such bioresorbable materials however are expensive to produce and sterilize. Further pharmaceutical active substances have to be used to afford a microbial action or in order also to prevent uncontrolled cell growth which in the least critical case causes at least undesirably severe scar formation.

Activation of and support for the body-specific healing mechanisms for rapid healing progress is also only very limitedly implemented in the conventional wound care methods.

A polymer material used as a bandage or wound covering material which contains antimicrobially active silver-bearing glass is also known. Elementary magnesium can be added to the material.

### SUMMARY OF THE INVENTION

An aspect of the present invention is to improve wound care by a biocompatible, biodegradable wound covering material. In that respect the invention seeks to provide that body-specific defense and healing mechanisms are supported and antimicrobial and antiproliferative effects are achieved.

In accordance with the invention that aspect is attained in that the wound covering material contains elementary magnesium. In that respect the elementary magnesium is present in the form of a biodegradable magnesium alloy. The wound to be treated is covered at least in part with magnesium in the form of an alloy or the magnesium is introduced at least in part into the wound in the form of an alloy.

“Biodegradation” denotes hydrolytic, enzymatic and other metabolism-governed breakdown processes in the living organism which lead to gradual dissolution of at least large parts of the materials used. The term biocorrosion is frequently used synonymously. The term bioresorption additionally includes subsequent resorption of the breakdown products.

It is generally known that magnesium ions are an indispensable and versatile activator of vital processes for life.

In regard to antimicrobial effects of magnesium ions it is known that the non-specific defense by way of the properdin system is effective only in the presence of magnesium ions and phagocytosis of bacteria by leucocytes experiences stimulation by magnesium. Accordingly, magnesium ions provide, inter alia, for combating infections by assisting or activating the body-specific immune system and also generally reduce susceptibility to infections.

In regard to wound healing magnesium ions are necessary for anaerobic metabolism and promote normal granulation of the connective tissue, that is to say, rapid wound healing by filling the wound with healthy tissue. In the case of burns, the presence of magnesium ions has a positive effect as it reduces the level of ammonia and thus promotes skin functions in the burn region.

The use of magnesium alloys (which contain elementary magnesium) in the context of wound care, in particular the direct application thereof, was unknown hitherto. Administration was hitherto typically effected in oral or intravenous form, generally as a nutritional supplement in order to support various body functions, but the magnesium was always present in ionic form.

### DETAILED DESCRIPTION OF THE INVENTION

It was now surprisingly found in in-vivo and in-vitro tests that the use of elementary magnesium in magnesium alloys of suitable composition counteracts strong immunological reactions on the part of the body. It was also possible to verify an antiproliferative effect in particular on smooth muscle cells and endothelium cells in in-vitro tests, that is to say, growths which are the cause of severe scarring effects could be averted or greatly checked, but controlled cell growth takes place.

The elementary magnesium is present in the form of a biodegradable magnesium alloy, by way of the composition of which it is possible to control the biodegradation behavior, that is to say in particular the degradation period, the mechanical properties and the biological effectiveness, as material investigations and the in-vivo and in-vitro tests have shown. The magnesium alloy should contain at least 50% by weight, preferably at least 70% by weight and particularly preferably at least 85% by weight of magnesium.

The operative mechanism which forms the basis for the positive effects has hitherto not been discovered in detail. Although not wishing to condition patent-ability on any particular theory, the correlation with the generally known effects of magnesium however point to the assumption that, in the direct use of elementary magnesium and magnesium alloys, the decomposition products which are absorbed upon degradation by the body also at least locally activate in part the same processes as the ionic magnesium which is absorbed for example with food. A direct antibacterial action is also not to be excluded. The alloy breakdown processes also appear to have an influence on what happens.

In accordance with a preferred variant of the above-indicated embodiment, the specific composition of the magnesium alloy and its modification are predetermined in such a way that decomposition starts immediately after application and is maintained until at least in part the wound is filled with connective tissue. That period of time should preferably be between 1 hour and 60 days, in particular between 1 and 10 days. The extent of the breakdown process is dependent on the conditions prevailing at the location of use.

It is conceivable for the antibacterial effect to be boosted by the deliberate addition of suitable metals such as for example silver, gold or rare earths, without worsening the biocompatibility and degradation properties.

Biodegradable magnesium alloys are particularly preferred, which contain rare earth metals and yttrium (WE-magnesium alloys), wherein the collective term “rare earth metal” is used to denote the elements scandium (atomic number 21), lanthanum (57) and the 14 elements following lanthanum cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71), referred to as lanthanides. In a particularly preferred feature, the magnesium alloys have the following proportions by weight of the alloy components:
rare earth metals between 2.0 and 5.0% by weight and/or
yttrium between 3.5 and 4.5% by weight and/or
neodymium between 1.5 and 3.0% by weight and/or
zirconium between 0.3 and 1.0% by weight and/or
aluminum <0.5% by weight, in particular <0.01% by weight and/or
balance <0.5% by weight, in particular <0.3% by weight,

wherein magnesium occupies the proportion by weight that remains to 100% by weight in the alloy. The above-mentioned magnesium alloys exhibit a favorable breakdown behavior, high biocompatibility of the alloy and also the breakdown products and have mechanical properties which are adequate for the area of use.

Different embodiments can be envisaged for efficient use. Depending on the respective wound size, depth and nature, powders or grains, threads or wires, cuttings or foils are to be preferred. Thus, powders or grains can be most easily used in relation to any wound sizes, threads and wire are possibly better suited to deep wounds while a foil is well suited for injuries of large area in which a good protective function in relation to external influences is also advantageous, as for example in the case of burn injuries. Instead of a foil it is also possible to use cloths which are woven or pressed from threads.

The magnesium-bearing material can be applied directly to the wound, it can be embedded into or applied to a further wound covering, or it can also be introduced into an ointment or flushing agent.

The magnesium-bearing material can also be used as a carrier material for further pharmaceutical active substances.

Direct contact of the magnesium-bearing material with the wound should be ensured however in order to achieve an optimum action on the part of the magnesium and separation of the wound and non-resorbable materials.

As magnesium and the magnesium alloys have a very high degree of transformation capability, processing by way of conventional material processing processes is not a problem. Bar, tube, cutting, wire or powder configurations can be obtained on the market (BDI-Einkaufsführer, Deutsche Industrie (“Federation of German Industries Purchasing Guide, German Industry”)). Conventional foils can be produced by way of rolling processes. Extremely thin magnesium foils (μm), in the past produced by way of hammering processes, can also be produced nowadays by way of powder coating processes.

Coatings on carrier materials can be produced for example by way of powder coating or sputtering.

The material can be sterilized in particular by means of gamma or beta radiation or also with alcohol solutions.

What is claimed is:

1. A wound covering material containing elementary magnesium characterized in that the magnesium is present in the form of a biodegradable alloy.

2. A wound covering material as set forth in claim 1, wherein the magnesium alloy is an alloy of type WE.

3. A wound covering material as set forth in claim 1, wherein a specific composition of the magnesium alloy and its modification is predetermined such that decomposition starts immediately after application and is maintained until the wound is covered with connective tissue at least region-wise.

4. A wound covering material as set forth in claim 1 characterized in that the magnesium-bearing material is in the form of a powder.

5. A wound covering material as set forth in claim 1 characterized in that the magnesium-bearing material is integrated into a wound dressing.

6. A wound covering material as set forth in claim 2, wherein a specific composition of the magnesium alloy and its modification is predetermined such that decomposition starts immediately after application and is maintained until the wound is covered with connective tissue at least region-wise.

7. A wound covering material as set forth in claim 1, wherein the alloy is at least 50 percent magnesium by weight.

8. A wound covering material as set forth in claim 2, wherein the alloy consists of magnesium and at least one component selected from the group consisting of 2.0 to 5.0 percent by weight rare earth element, 3.5 to 4.5 percent by weight yttrium, 1.5 to 3.0 percent by weight neodymium, 0.3 to 1.0 percent by weight zirconium, less than 0.5 percent by weight aluminum, and less than 0.5 percent by weight other material.
